(19) United States
(12) Patent Application Publication
(10) Pub. No.: US 2024/0091035 A1
(43) Pub. Date: Mar. 21, 2024

(54) BRAIDED STENT

(71) Applicant: KONINKLIJKE PHILIPS N.V., EINDHOVEN (NL)

(72) Inventors: Scott Robert WILSON, MAPLE GROVE, MN (US); Cedar DECKER, ALBERTVILLE, MN (US); Jake MERTENS, MAPLE GROVE, MN (US)

(21) Appl. No.: 18/266,019

(22) PCT Filed: Dec. 14, 2021
(86) PCT No.: PCT/EP2021/085577

§ 371 Date: Jun. 8, 2023

Related U.S. Application Data
(60) Provisional application No. 63/126,023, filed on Dec. 16, 2020.

(51) Int. Cl. A61F 2/90; A61L 31/02; A61L 31/08; A61L 31/18

(52) U.S. Cl. CPC A61F 2/90; A61L 31/022; A61L 31/088; A61L 31/18; A61F 2210/0014; A61F 2210/0076; A61F 2220/0058; A61F 2250/0098

(57) ABSTRACT

A stent (10) includes a hollow tube (12) including interlaced metal strands (14); and a reinforcement providing radial strength reinforcement at an end (16, 18) of the hollow tube. In some examples, the reinforcement includes a first reinforcement at a first end (16) of the hollow tube (12); and a second reinforcement at a second end (18) of the hollow tube opposite the first end of the hollow tube.

### FIELD

The following relates generally to the stent arts, stent manufacturing arts, stent assembly arts, and related arts.

### BACKGROUND

Stents constructed as self-expandable, metal support structures, delivered via intravascular devices, are commonly used in the treatment in intravascular disease, as well as in larger regions of the anatomy such as the esophagus. Self-expanding stents are typically made from a braided wire mesh or from laser cut tubes. Braided stents typically are made from a plurality of wires, spiral wound into a braided tubular structure. These stents are manufactured using a braiding machine (e.g., available from Steeger USA Inc., Inman, South Carolina, USA), and are manufactured in long lengths, then cut to size, leaving open wire ends. An example of a stent of this type is the WallStent Endoprosthesis Stent, available from Boston Scientific, Marlborough, Massachusetts, USA. Stents are placed in the vascular system or esophagus to expand the vessel or esophageal diameter to treat various disease states.

The following discloses certain improvements to overcome these problems and others.

### SUMMARY

In some embodiments disclosed herein, a stent includes a hollow tube comprising interlaced metal strands; and a reinforcement providing radial strength reinforcement at an end of the hollow tube.

In some embodiments disclosed herein, a method of assembling a stent includes braiding a plurality of metal strands to form a hollow tubular body; heat setting the hollow tubular body; and after the heat setting, forming a reinforcement providing radial strength reinforcement at one or both ends of the hollow tubular body.

One advantage resides in providing a stent with closed or fixed ends that provide greater radial strength at the open ends of the stent.

Another advantage resides providing in a stent with an optimal wire pitch or pic rate (per inch crossings) that provide improved conformability.

Another advantage resides in providing a stent made from Nitinol for improved durability.

Another advantage resides in providing a stent with an optimal braid angle at one or more ends of the stent.

Another advantage resides in providing a stent having enhanced radiopacity.

Another advantage resides in providing a stent with a variable radial strength along a length thereof.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a stent in accordance with the present disclosure.

FIGS. 2, 3, 4, 5, 6A, 6B, 6C, and 7 diagrammatically illustrates reinforcements of the stent of FIG. 1.

FIG. 8 diagrammatically illustrates a method of assembling the stent of FIG. 1.

### DETAILED DESCRIPTION

Braided stents by design tend to be more durable and flexible compared to laser cut stents. However, one limitation of braided stents is the radial strength at the open ends of the stent. The unrestricted movement of the wire ends results in lower radial strength/crush resistance at the ends of the stent, compared to the mid-section of the stent. This can result in poor outcomes due to reduced patency of the stent. In addition, commercially available braided stents tend to have lower radial force that equivalent laser cut stents.

The following relates to improvements in a braided stent. Such stents are typically manufactured by braiding wires made of Nitinol (a metal alloy of nickel and titanium where the Ni and Ti are present in roughly equal atomic percentages) on a mandrel and then heat setting the braided stent at typically 400-500° C. Other materials may also be used, such as platinum, titanium, gold, silver, stainless steel, or combination materials (e.g., a drawn-filled tube wire such as a Nitinol Drawn Filled Tube (available from DFT Wire, Fort Wayne Metals, Indiana, USA), which has an inner core of one or more radiopaque materials (i.e., platinum, tantalum, etc.). The resulting braided stent is self-expanding, so that it can be radially compressed to fit into a catheter tip or other delivery instrument and then expands into position in the vein when placed.

However, as noted, existing braided stents of this type suffer from reduced crush resistance and reduced radial strength at the ends of the stent. This is recognized herein to be due at least in part to loose wire ends at the ends of the stent. In view of this, the following discloses various approaches for increasing crush resistance and radial strength at the stent ends.

In some embodiments disclosed herein, the wire ends of a braided stent are bent over and connected to neighboring wire ends to form closed loops. In one approach, after the first heat set step, the Nitinol wire ends are bent to form the closed loops and a second heat set step is performed. Before or after the second heat set step, the formed wire ends of are secured together by welding (e.g., laser welding) or using crimp sleeves. This approach has an additional advantage of eliminating the loose wire ends, which reduces the possibility of the loose wire ends embedding into the blood vessel with potentially detrimental effects such as providing nucleation sites for thrombus or atherosclerosis.

In other embodiments disclosed herein, the wire crossings near the ends are welded together (e.g., by laser welding) to increase crush resistance and radial strength. This could be done alone, or in combination with the forming of closed loop ends as previously described.

In some embodiments disclosed herein, the wire ends are encapsulated with a polymer encapsulant by dip coating, spray coating, or another approach. Depending on the encapsulant material, a cure step may be added to cure the polymer. In some examples, the encapsulant covers the ends including filling in gaps in the braid. In another example, the braid gaps are not filled by the encapsulant. It is also contemplated to coat the entire stent rather than just the ends. This approach can optionally be combined with one or both of the previously described approaches.

In other embodiments disclosed herein, the braid density is increased at the ends. This can be measured by braid angle, or equivalently by the pics per inch (PPI). In one embodiment, the braid pitch or braid angle is increased by 25% or more at the ends compared with the central portion of the stent. In another (not necessarily mutually exclusive) embodiment, the braid has PPI=15 or higher at the ends. This approach can optionally be combined with one or more of the earlier-described approaches.

In some embodiments disclosed herein, the disclosed approaches can be deployed at both ends of the stent, or in a variant embodiment only at one end of the stent. The latter approach may be appropriate if, for example, the stent placement is known to lead to higher compression at one end of the stent as compared with the other end of the stent.

In other embodiments disclosed herein, a radiopaque marker can be placed at one or both ends of the stent. This may be gold or any other material that is absorbing for X-rays used in X-ray imaging. For example, a gold wire may be wrapped around each end. In the closed loop embodiment described previously, the crimp sleeves could be made of a radiopaque material to provide this imaging benefit as a secondary advantage. a

While described in the context of venous stents, the disclosed approaches are suitable for use in arterial stents and other types of stents such as esophageal stents. While Nitinol is the preferred material for these types of braided stents, braided stents of metal wires with high elasticity could be used instead.

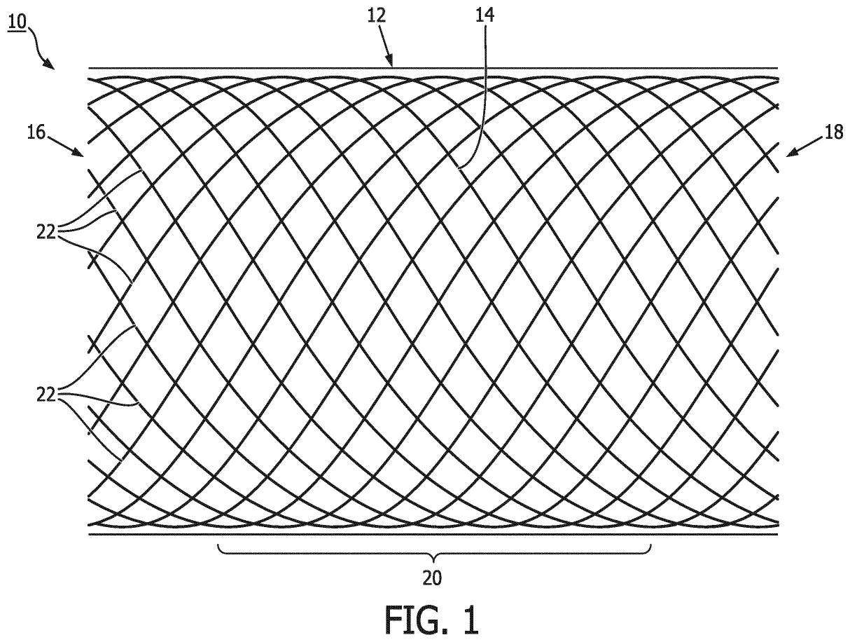

With reference to FIG. 1, an illustrative stent 10 (e.g., an arterial, venous, or esophageal stent) is shown. As shown in FIG. 1, the stent 10 comprises a hollow tube or tubular structure 12 made from a plurality of interlaced metal or wire strands 14. The metal strands 14 can be made from Nitinol, or any other suitable material with high elasticity. The hollow tube 12 includes a first (i.e., left) end portion 16, a second, opposing (i.e., right) end portion 18, and a middle portion 20 interspersed therebetween. In addition, each metal strand 14 includes opposing ends located at the ends 16, 18 of the stent 10.

The stent 10 also include a reinforcement that provides radial strength reinforcement at one or more of the end portions 16, 18 of the hollow tube 12. For example, as shown in FIG. 1, the reinforcement comprises ends of the metal strands 14 at the first end portion 16 being formed into loops 22 secured to neighboring metal strands 14. The Nitinol of the metal strands 14 allows the loops 22 to be formed, and then the hollow tube 12 is heat treated to maintain these desired shapes. The loops 22 can be formed at the first end portion 16 (as illustrated), or at the second end portion 18, or at both ends of the stent. While the illustrative stent 10 has a constant diameter along its length, alternatively the stent 10 can be tapered to conform to a tapered diameter of a native vessel into which the stent 10 is deployed.

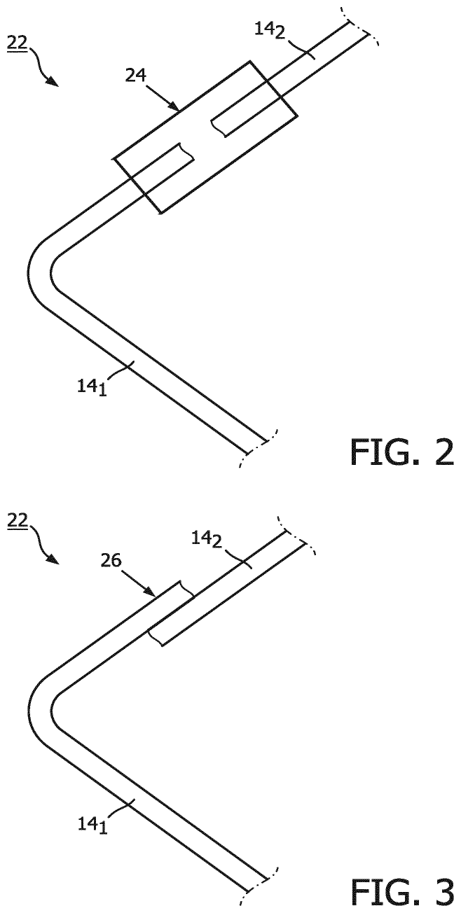

FIG. 2 shows a zoomed view of the end of one of the metal strands 141 of the hollow tube 12 that is bent to join the end of another metal strand 142 of the hollow tube 12. As shown in FIG. 2, the ends of the two metal strands 141, 142 are crimped together with a crimp sleeve 24. In this example, the combination of the loops and the crimp sleeves 24 can comprise the reinforcement. The crimp sleeves 24 can be used to crimp the ends of the metal strands 14 together to form the loops 22.

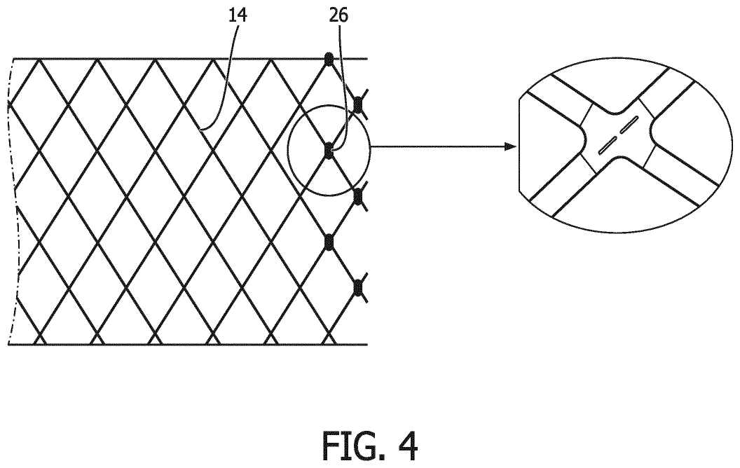

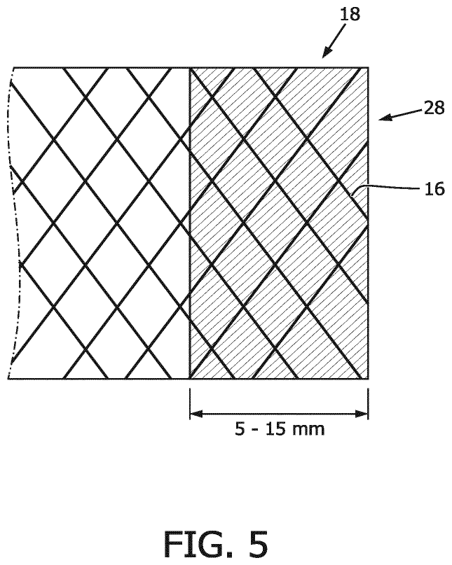

FIG. 3 shows another zoomed view of the end of one of the metal strands 141 of the hollow tube 12 that is bent to join the end of another metal strand 142 of the hollow tube 12. As shown in FIG. 3, the ends of the two metal strands 141, 142 are welded together with a weld 26. In this example, the combination of the loops and the welds 26 joining together the ends forming the loops can comprise the reinforcement. The welds 26 can be used to secure the loops 22 formed in the ends of some wires 141 to the ends of neighboring metal strands 142. As shown in FIG. 1, the weld 26 can be disposed at one of the end portions 16, 18. In another example, shown in FIG. 4, the welds 26 can additionally or alternatively bond crossings of two of the interlaced metal strands 14 of the hollow tube 12. The welds 26 of FIGS. 3 and 4 can, for example, comprise laser welds. Other metal joinders could also be used in place of the welds 26, such as solder bonds formed using a biocompatible solder material or brazed bonds formed using a biocompatible filler material.

Reinforcement by way of forming the loops 22 at one or both ends 16, 18 of the stent 10 advantageously provides radial strength reinforcement at the end(s). Additionally, the loops 22 eliminate unsecured ends of the wires 14 at the end(s) of the stent, by bonding ends of neighboring wires (e.g., the ends of illustrative wires 141, 142) together. This can be advantageous because the loops 22 are less likely to abrade or embed into the inner blood vessel wall. Such abrasion or embedding of the wire ends can provide potential nucleation sites for thrombus or atherosclerosis, and/or can weaken the blood vessel wall.

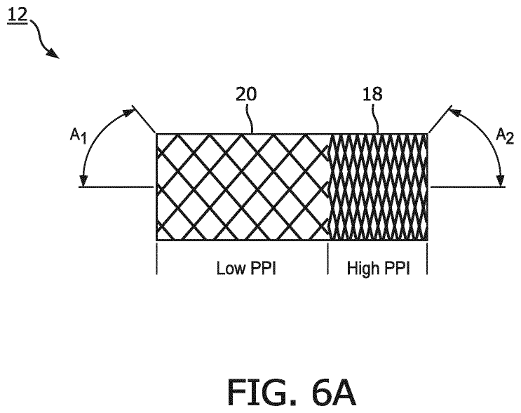

FIG. 5 shows another example of a reinforcement, this time shown located at the right end 18 of the stent 10 (again, the reinforcement can be at either end 16, 18 or at both ends 16 and 18). As shown in FIG. 5, the reinforcement can comprise an encapsulant 28 disposed over one or more of the end portions 16, 18 of the hollow tube 14. The encapsulation 28 can comprise a polymer, such as a low durometer silicone or urethane, which can provide securement of the metal strands 14, but still flexible to allow the hollow body 12 to be crimped into a delivery system. As shown in FIG. 5, the polymer coating is applied to one of the end portions 16, 18 for a length of approximately 5-15 mm. In one example, the interlaced metal strands 14 form a mesh with openings, and the encapsulant 28 does not fill the openings of the mesh at the end portion 16, 18 of the hollow tube 12. In another example, the encapsulant 28 does fill the openings in the mesh. Whether the openings in the mesh are filled depends on the amount of encapsulant material applied and the method of its application. For example, if a relatively thin layer of encapsulant is applied by spray coating then it will generally not fill the mesh openings; whereas, if a thicker layer is applied by dip coating then the mesh openings may be (at least partially) filled in by the encapsulant material.

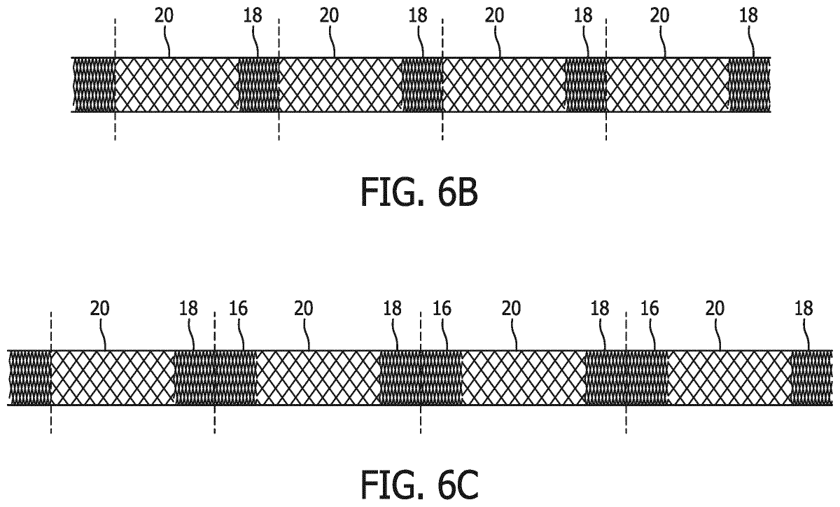

FIG. 6A shows another example of a reinforcement. As shown in FIG. 6A, the reinforcement comprises a braid pitch A2 at one or both of the end portions 16, 18 of the hollow tube 12 (specifically at the right end portion 18 in illustrative FIG. 6A) that is greater than a braid pitch A1 at the middle portion 20 of the hollow tube 12. For example, the braid pitch A2 at the end portion(s) 16, 18 of the hollow tube 12 is at least 25% greater than the braid pitch A1 at the middle portion 20 of the hollow tube 12, as measured for example by the braid angle or in units of PPI (pics per inch). The braid pitch can directly impact the radial strength and conformability of the stent 10. A higher braid angle or PPI results in a tighter wound braid, increasing the radial strength and increasing the ability of the stent 10 to conform to a curve. However, the higher braid pitch may also increase a change in length of the stent 10 from its compressed state to its expanded state, making delivery of the stent 10 more variable. Hence, it is advantageous to employ the higher braid pitch A2 at one or both ends 16, 18 of the stent 10 to strengthen the end(s) which are most susceptible to deformation or crushing due to the reduced radial strength at the ends, while using the lower braid pitch A1 in the middle portion 20 of the stent 10 to retain delivery repeatability of the stent 10 within the blood vessel. A non-limiting illustrative braid pattern for the stent 10 can have a PPI of 12-18 (with a target of 15, and a braid angle A2 of approximately 55-65 degrees). This allows the stent 10 to conform to the anatomy, with sufficient radial force to treat disease. A typical stent can have a PPI of 10-11, and A1 can have angle of 110°-130°.

As shown in FIG. 6A, the PPI could be varied along the length of the hollow tube 12. For example, the hollow tube 12 could have a higher PPI at one or both of the end portions 16, 18 of the hollow tube 12 to provide higher radial strength, and a lower PPI along the middle portion 20 of the hollow tube 12. The higher PPI at the end portion(s) 16, 18 can improve the securement of the stent 10 to the vessel wall, preventing migration of the stent 10.

With reference to FIG. 6B, a suitable approach for manufacturing the stent 10 with higher braid pitch at the right end 18 is shown. Braided stents typically are made from a plurality of wires, which are spiral wound into a braided tubular structure using a braiding machine in long lengths. Individual stents 10 of a desired length are then formed by cutting this long length of braided tube to size. As shown in FIG. 6B, this entails running the braiding machine to increase the braid pitch for a length portion corresponding to the length of the right end 18, with these portions of higher braid pitch spaced apart at intervals corresponding to the intended length of the stents. Then, the stents are cut from the long tube with the regions 18 of higher braid pitch at the (e.g., illustrative) right ends of the cut stents. In FIG. 6B, the cut locations are indicated by vertical dashed lines.

With reference to FIG. 6C, a suitable approach for manufacturing the stent 10 with higher braid pitch at both left and right ends 16, 18 is shown. Here, the braiding machine is run to increase the braid pitch for a length portion corresponding to the combined lengths of the left and right ends 16, 18, with these portions of higher braid pitch again spaced apart at intervals corresponding to the intended length of the stents. Then, the stents are cut from the long tube with the cut locations (again indicated by vertical dashed lines in FIG. 6C) located at the middles of regions of higher braid pitch. In this way, the part of the higher-pitch portion to the left of the cut forms the higher braid pitch right end 18 of the stent to the left of the cut, and the higher-pitch portion to the right of the cut forms the higher braid pitch left end 16 of the stent to the right of the cut.

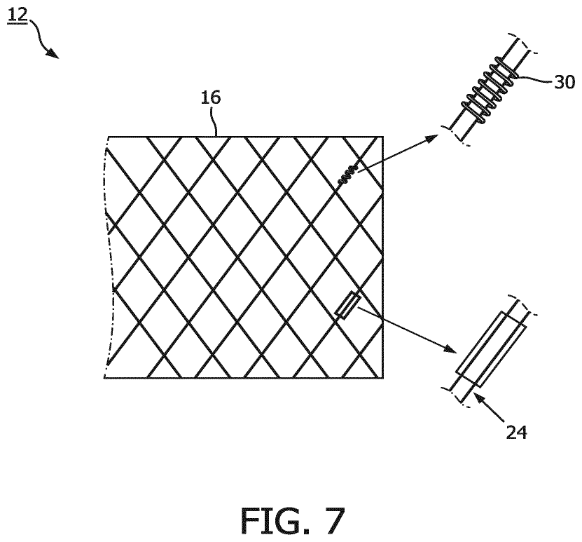

FIG. 7 shows that the stent can include radiopaque markers. Radiopacity of stent 10 is important for visibility during x-ray or fluoroscopy procedures. To enhance the radiopacity of the stent 10, radiopaque markers made from high molecular weight materials (e.g., platinum, tantalum, gold, etc.) could be placed at desired locations on the stent. In one example, as shown in FIG. 7, the markers can comprise the crimp sleeves 24 being made from a radiopaque material, such as a gold coating, which is absorbing for X-rays. In another example, the markers can comprise a wire coil 30 made from a gold coating. In yet another variant embodiment, if the welds 26 of the embodiment of FIG. 3 are replaced by metal joinders comprising solder bonds or braised bonds, then the solder material or the filler material used in forming the braised bonds could be a radiopaque material.

It will be appreciated that the stent 10 can include multiple examples of the reinforcements described above. For example, the stent 10 can include both the loops 22 and the encapsulant 28, or the encapsulant 28 and the welds 26, and so forth. In addition, the first end portion 16 can include a first reinforcement (e.g., the loops 22), and the second end portion 18 can include a second reinforcement (e.g., the encapsulant 28), or both end portions 16, 18 can include the same reinforcement (e.g., loops 22 at both end portions 16, 18). These are merely illustrative examples and should not be construed as limiting.

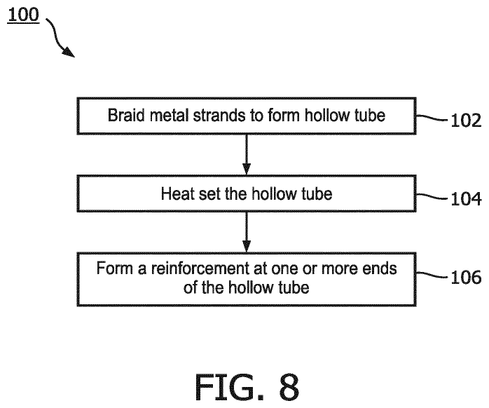

FIG. 8 shows an example of a flowchart showing a method 100 of assembling the stent 10. At an operation 102, a plurality of metal strands 14 are braided to form a hollow tubular body 12. At an operation 104, the hollow tubular body 12 is heat set. At an operation 106, after the heat setting operation 104, a reinforcement is formed in or on the hollow tubular body 12. The reinforcement provides radial strength reinforcement at one or both end portions 16, 18 of the tubular body 12.

The reinforcement operation 106 can be performed in a variety of manners. In one example, the forming of the reinforcement structure includes bending ends of the metal strands 14 at one or both end portions 16, 18 of the tubular hollow body 12 to form one or more loops 22. A second heat setting operation can be performed to heat set the loops 22. In another (non-mutually exclusive) example, one or more crimp sleeves 24 can be applied to the ends of the metal strands 14. In another (non-mutually exclusive) example, one or more welds 26 can be formed on the ends of the metal strands 14 to secure the loops 22 to neighboring metal strands 14. In another (non-mutually exclusive) example, an encapsulant 28 can be added to one or both end portions 16, 18 of the tubular structure 12. To do so, the encapsulant 28 can be applied to one or both end portions 16, 18 of the tubular structure 12 by dip coating or spray coating.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A stent, comprising:
a hollow tube comprising interlaced metal strands; and
a reinforcement providing radial strength reinforcement at an end of the hollow tube.

2. The stent of claim 1, wherein the reinforcement comprises ends of the metal strands at the end of the hollow tube which are formed into loops secured to neighboring metal strands.

3. The stent of claim 2, wherein the reinforcement further comprises crimp sleeves securing the loops to the neighboring metal strands.

4. The stent of claim 3, wherein the crimp sleeves are comprised of a radiopaque material that is absorbing for X-rays.

5. The stent of claim 4, wherein the radiopaque material comprises a gold coating of the crimp sleeves.

6. The stent of claim 2, wherein the reinforcement further comprises welds securing the loops to the neighboring metal strands.

7. The stent of claim 1, wherein the reinforcement comprises welds that bond crossings of the interlaced metal strands of the hollow tube.

8. The stent of claim 6, wherein the welds comprise laser welds.

9. The stent of claim 1, wherein the reinforcement comprises an encapsulant disposed over the end of the hollow tube.

10. The stent of claim 9, wherein the interlaced metal strands form a mesh with openings and the encapsulant disposed over the end of the hollow tube does not fill the openings of the mesh at the end of the hollow tube.

11. The stent of claim 9, wherein the interlaced metal strands form a mesh with openings and the encapsulant fills the openings of the mesh at the end of the hollow tube.

12. The stent claim 1, wherein the reinforcement comprises a braid pitch at the end of the hollow tube that is greater than a braid pitch at a middle of the hollow tube.

13. The stent of claim 12, wherein the braid pitch at the end of the hollow tube is at least 25% greater than the braid pitch at the middle of the hollow tube.

14. The stent of claim 1, wherein the metal strands are comprised of nitinol.

15. The stent of claim 1, wherein the reinforcement includes:
a first reinforcement at a first end of the hollow tube; and
a second reinforcement at a second end of the hollow tube opposite the first end of the hollow tube.

16. A method of assembling a stent, the method comprising:
braiding a plurality of metal strands to form a hollow tubular body;
heat setting the hollow tubular body; and
after the heat setting, forming a reinforcement providing radial strength reinforcement at one or both ends of the hollow tubular body.

17. The method of claim 16, wherein the forming of the reinforcement includes:
bending ends of the metal strands at the one or both ends of the hollow tubular body to form loops; and
performing a second heat setting to heat set the loops.

18. The method of claim 17, wherein the forming of the reinforcement further includes applying crimp sleeves or performing welding to secure the loops to the neighboring metal strands.

19. The method of claim 16, wherein the forming of the reinforcement includes encapsulating the one or both ends of the hollow tubular body with an encapsulant.

20. The method of claim 19, wherein the encapsulating comprises applying the encapsulant to the one or both ends of the hollow tubular body by dip coating or spray coating.
